(19) United States
(12) Patent Application Publication
(10) Pub. No.: US 2020/0143947 A1
(43) Pub. Date: May 7, 2020

(54) DEVICE AND METHOD FOR MEASURING RISK OF DRY EYE, AND COMPUTER PROGRAM FOR EXECUTING METHOD

(71) Applicant: SAMSUNG MEDICAL CENTER, Seoul (KR)

(72) Inventor: Chul Young CHOI, Seoul (KR)

(21) Appl. No.: 16/736,523

(22) Filed: Jan. 7, 2020

Related U.S. Application Data
(63) Continuation of application No. PCT/KR2018/007676, filed on Jul. 6, 2018.

(30) Foreign Application Priority Data
Jul. 7, 2017 (KR) 10-2017-0086612

(51) Int. Cl. G16H 50/30; G06K 9/00; A61B 3/10; A61B 5/00

(52) U.S. Cl. CPC G16H 50/30; A61B 5/7275; A61B 3/101; G06K 9/0061

(57) ABSTRACT

The present application relates to a method for measuring the risk of dry eye. The method may include receiving first sensing data formed by sensing a set area in a body of a user and receiving first risk factor information related to the user. The method may also include outputting first blink information related to blinking of the user, on the basis of the first sensing data and calculating a first weighted value corresponding to the first risk factor information, on the basis of a relationship between one or more pieces of the received first risk factor information and a risk degree. The method may further include calculating a first risk of dry eye of the user by combining the first blink information and the first weighted value, and providing the first risk of dry eye through an input/output device.

### CROSS-REFERENCE TO RELATED APPLICATIONS

This application is a continuation application and claims the benefit under 35 U.S.C. §§ 120 and 365 of PCT Application No. PCT/KR2018/007676, filed on Jul. 6, 2018, which is hereby incorporated by reference. PCT/KR2018/007676 also claims priority to Korean Patent Application No. 10-2017-0086612 filed on Jul. 7, 2017 which is hereby incorporated by reference.

### BACKGROUND

### Technical Field

An aspect of the present disclosure relates to a device and a method for measuring the risk of dry eye, and a computer program for executing the method, and more particularly, to a device, a method, and a computer program for measuring the risk of dry eye of a user by combining pattern information related to sensed blinks with biometric information related to the user, environmental information related to the user, and habit information related to the user.

### Related Technology

Many smartphone users currently suffer from dry eye syndrome that is characterized by the symptoms of eye strain, bleariness, discomfort, redness, vision deterioration, and the like. Dry eye syndrome is not simply a disease in which the amount of tears is insufficient, but the instability of a tear film caused by inflammation of the surface of an eye and a disorder of a healthy tear structure such as a mucin layer or the like is accepted as the definition thereof. Recently, this has also been closely related to VDT syndrome, and the unusually rapid decrease in the number of blinks when focusing on something is considered to be one of trigger factors thereof. In normal individuals, reduced blink patterns of 5 times or less per minute that persist for a preset time a day may depend on multiple factors of the individuals but may develop into an advanced form of dry eye syndrome that may cause discomfort, dryness, and even visual deterioration.

In addition, the development of smartphone technology has advanced enough to incorporate facial recognition, iris recognition, and eye blinks into user authentication technology.

### SUMMARY

Provided are an electronic device and a method for predicting an inherent risk of dry eye of a user by combining blink pattern information sensed from the user and biometric information, environmental information, eye disease information, and medical history information of the user.

Provided are also an electronic device and a method set to receive guide information capable of reducing a risk of dry eye of a user and restrict driving of a screen according to the guide information.

Provided are also an electronic device and a method for determining an improvement effect of guide information by remeasuring a risk of dry eye of a user after restricting driving according to the guide information.

A method of measuring the risk of dry eye according to aspects of the present disclosure, may include: receiving first sensing data formed by sensing a set area in a body of a user; receiving first risk factor information related to the user; outputting first blink information related to blinking of the user, on the basis of the first sensing data; calculating a first weighted value corresponding to the first risk factor information, on the basis of a relationship between one or more pieces of received risk factor information and a risk degree; and calculating a first risk of dry eye of the user by combining the first blink information and the first weighted value, and providing the first risk of dry eye through an input/output device.

According to the aspects, the method of measuring the risk of dry eye may further include generating and providing a message indicating the first risk of dry eye.

The method of measuring the risk of dry eye according to the aspects of the present disclosure, may further include: determining whether the first risk of dry eye belongs to a first reference range; on the basis of the determination result, transmitting a request message including the first risk of dry eye to an expert group connected to the user or the first reference range; and receiving and providing a response message, which is a response to the request message, including guide information capable of reducing the first risk of dry eye.

The method of measuring the risk of dry eye according to the aspects of the present disclosure may further include, when the response message is received, separating the guide information included in the response message and setting the guide information in a device.

The guide information may include at least one selected from a longest continuous use time, screen brightness information, and rest time information after continuous use.

The method of measuring the risk of dry eye according to the aspects of the present disclosure may include: after the setting, receiving second sensing data formed by sensing the set area and receiving second risk factor information at a point in time after the terminal use restriction is applied; calculating a second weighted value corresponding to the second risk factor information; outputting second blink information related to the blinking of the user, on the basis of the second sensing data; and calculating a second risk of dry eye of the user by combining the second blink information and the second weighted value, generating an improvement effect of the guide information by comparing the first and second risks of dry eye, and providing a message including the improvement effect.

A device for measuring the risk of dry eye according to aspects of the present disclosure may include: a sensing data receiver configured to receive first sensing data formed by sensing a set area in a body of a user; a risk factor information receiver configured to receive first risk factor information related to the user; a data converter configured to output first blink information related to blinking of the user, on the basis of the first sensing data; a weighted value calculator configured to calculate a first weighted value corresponding to the first risk factor information, on the basis of a relationship between one or more pieces of received risk factor information and a risk degree; a risk calculator configured to calculate a first risk of dry eye of the user by combining the first blink information and the first weighted value; and a message provider configured to provide a message including the first risk of dry eye through an input/output device.

A computer program according to an aspect of the present disclosure may be stored in a medium to execute any one of the methods according to the aspects of the present disclosure by using a computer.

Provided is also a computer-readable recording medium having recorded thereon a computer program for executing another method, another system, and the method for embodying the present disclosure.

Other aspects, features, and advantages other than those described above will become more apparent from drawings, claims, and detailed description of the disclosure that follow below.

According to the present disclosure, an inherent risk of dry eye of a user may be predicted by combining the number of blinks sensed from the user and biometric information, environmental information, and habit information of the user.

Also, according to the present disclosure, guide information capable of reducing a risk of dry eye of the user may be received, and driving of a screen may be restricted according to the guide information.

In addition, according to the present disclosure, an improvement effect of the guide information may be determined by remeasuring a risk of dry eye of the user after restricting the driving according to the guide information.

### BRIEF DESCRIPTION OF THE DRAWINGS

FIG. 1 is a block diagram of a medical system according to an aspect of the present disclosure.

FIG. 2 is a block diagram of a device for measuring the risk of dry eye according to an aspect of the present disclosure.

FIG. 3 is a block diagram of a storage medium.

FIGS. 4 through 7 are flowcharts of a method for measuring the risk of dry eye according to aspects of the present disclosure.

### DETAILED DESCRIPTION

The present disclosure may include various embodiments and modifications, and exemplary embodiments thereof will be illustrated in the drawings and will be described herein in detail. The effects and features of the present disclosure and the accompanying methods thereof will become apparent from the following description of the embodiments, taken in conjunction with the accompanying drawings. However, the present disclosure is not limited to the embodiments described below, and may be embodied in various modes.

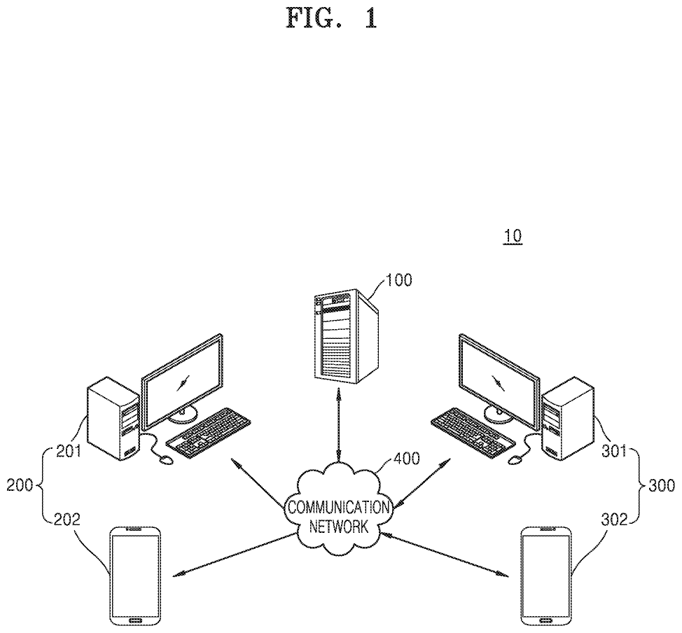

FIG. 1 is a schematic view illustrating a medical system 10 according to aspects of the present disclosure.

Referring to FIG. 1, the medical system 10 according to an aspect of the present disclosure may include a medical service providing server 100, a device 200 for measuring a risk of dry eye, an electronic device 300 of an expert group, and a communication network 400.

When receiving a signal including a risk of dry eye from the device 200, the medical service providing server 100 may transmit the signal including the risk of dry eye to an expert group that is determined in consideration of a user and the risk of dry eye. The signal including the risk of dry eye is transmitted to an expert group corresponding to the risk of dry eye or an expert group corresponding to each user. The medical service providing server 100 may perform a function of transmitting logic and algorithm for measuring the risk of dry eye to the device 200. The medical service providing server 100 may transmit guide information received from the electronic device 300 of the expert group to the device 200 for measuring a risk of dry eye of a user corresponding to the guide information. The medical service providing server 100 may transmit result data for the guide information received by the device 200 to the electronic device 300 of the corresponding expert group.

As described above, the medical service providing server 100 may manage data transmission and reception between the device 200 and the electronic device 300 of the expert group. Therefore, the user may simply measure the risk of dry eye and may receive simple medical information by which the risk of dry eye is lowered, by using the device 200.

The user may be provided with medical services through the device 200 and the electronic device 300. Also, the expert group may remotely provide medical services through the electronic device 300.

A plurality of devices 200 for measuring a risk of dry eye or a plurality of electronic devices 300 refer to communication terminals capable of using web services in wired and wireless communication environments. Here, the devices 200 or the electronic devices 300 may be personal computers 201 and 301 of a user or portable terminals 202 and 302 of the user. The portable terminals 202 and 302 are illustrated as smartphones in FIG. 1, but the spirit of the present disclosure is not limited thereto. Therefore, any terminal loading thereinto an application embodied to provide medical services and measure a risk of dry eye as described above may be used without limitation.

In more detail, the devices 200 or the electronic devices 300 may include computers (e.g., desktop, laptop, tablet, or the like), media computing platforms (e.g., cable, satellite set-top boxes, or digital video recorders), handheld computing devices (e.g., PDA, e-mail clients, or the like), any forms of cellphones, or any forms of other types of computing or communication platforms, but the present disclosure is not limited thereto.

The communication network 400 connects the plurality of devices 200 or the plurality of electronic devices 300 and the medical service providing server 100. In other words, the communication network 400 refers to a communication network providing a connection path through which the devices 200 or the electronic devices 300 are connected to the medical service providing server 100 and transmit and receive data. The communication network 400 may, for example, include wired networks such as Local Area Networks (LANs), Wide Area Networks (WANs), Metropolitan Area Networks (MANs), Integrated Service Digital Networks (ISDNs), or the like or wireless networks such as wireless LANs, CDMA, Bluetooth, satellite communications, or the like, but the scope of the present disclosure is not limited thereto.

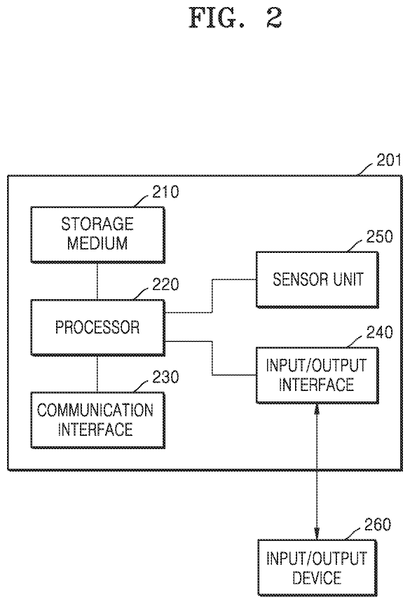

FIG. 2 is a block diagram illustrating a structure of a device 201 for measuring a risk of dry eye to improve eye health of a user according to an aspect of the present disclosure.

FIG. 2 illustrates the structure of the device 201 for measuring a first risk of dry eye as an example of one device for measuring a risk of dry eye. A structure of a device 202 for measuring a risk of dry eye may be the same as the structure of the device 201 for measuring the first risk of dry eye.

The device 201 may include a storage medium 210, a processor 220, a communication interface 230, an input/output interface 240, and a sensor unit 250. The storage medium 210 may be a computer-readable recording medium and may include a permanent mass storage device, such as random access memory (RAM), read only memory (ROM), and a disk drive. In addition, the storage medium 210 may store an operating system and at least one program code (e.g., a code for a browser installed and driven in the device 201, an application for providing a particular service, or the like). These software components may be loaded from a computer-readable recording medium separate from the storage medium 210 by using a drive mechanism. This separate computer-readable recording medium may include a computer-readable recording medium such as a floppy drive, a disk, a tape, a DVD/CD-ROM drive, a memory card, or the like. In another aspect, the software components may be loaded into the storage medium 210 through the communication interface 230 not into a computer-readable recording medium. For example, at least one program may be loaded into the storage medium 210 on the basis of a program (e.g., the above-described application) installed by a file provided through the communication network 400 by developers or a file distribution system (e.g., the medical service providing server 100 described above) for distributing an installation file of an application.

The processor 220 may process an instruction of a computer program by performing basic arithmetic, logic, and input/output operations. The instruction may be provided for the processor 220 by the storage medium 210 or the communication interface 230. For example, the processor 220 may execute a received instruction according to a program code stored in a recording device such as the storage medium 210.

The communication interface 230 may provide a function to allow the device 201 and the medical service providing server 100 to communicate with each other through the communication network 400 and may provide a function to communicate with another electronic device (e.g., the device 202) or another server. For example, a request (e.g., a request for a messaging service) generated by the processor 220 of the device 201 according to a program code stored in a recording device such as the storage medium 210 may be transmitted through the communication network 400 to the medical service providing server 100 under control of the communication interface 230. On the contrary, a control signal or instruction, contents, a file, or the like provided under control of a processor (not shown) of the medical service providing server 100 may be received through the communication interface 230 of the device 201 by the device 201 after passing through a communication interface and the communication interface network 400. For example, the control signal or instruction of the medical service providing server 100 received through the communication interface 230 may be transmitted to the processor 220 or the storage medium 210, and the contents, the file, or the like may be stored in a storage medium that may be further included in the device 201 for measuring the first risk of dry eye.

The input/output interface 240 may be a means for interfacing with an input/output device 260. For example, an input device may include a device such as a keyboard, a mouse, or the like, and an output device may include a device such as a display for displaying a communication session of an application. As another example, the input/output interface 240 may be a means for interfacing with a device in which functions for input and output are combined into one like a touch screen. In more detail, when the processor 220 of the device 201 processes the instruction of the computer program loaded into the storage medium 210, a service screen or contents formed by using data provided by the medical service providing server 100 or the device 202 may be displayed on a display through the input/output interface 240.

The sensor unit 250 is an element for sensing eyeball movements of the user. The sensor unit 250 may include an image sensor. The image sensor senses the eyeball movements of the user by pre-programmed instructions. The sensor unit 250 first extracts an eyeball of the user from an acquired image frame. When the eyeball of the user is not detected from the image frame, the sensor unit 250 may change a capturing position. When an eye is not detected from a facial area of the user, the sensor unit 250 may further perform a process of resetting the facial area. Here, the sensor unit 250 may set the facial area to a wider range than a first facial area and detect an eye from the set facial area. Even if an entire area is set to the facial area, when a position of the eye is not detected, the sensor unit 250 may provide the user with a notification of resetting the facial area. Selectively, when the position of the eye is not detected, the sensor unit 250 stops calculating a risk of dry eye.

Also, in other aspects, the device 201 and the medical service providing server 100 may include more elements than the elements of FIG. 2. However, most existing elements do not need to be clearly shown. For example, the device 201 may be embodied to include at least a part of the input/output device 260 described above or may further include other types of elements such as a transceiver, a Global Positioning System (GPS) module, a camera, various types of sensors, a database, and the like. In more detail, when the device 201 is a smartphone, the device 201 may be embodied to further include various types of elements such as an acceleration sensor or a gyro sensor, a camera, various types of physical buttons, buttons using a touch panel, an input/output port, a vibrator for vibration, and the like that are generally included in the smartphone.

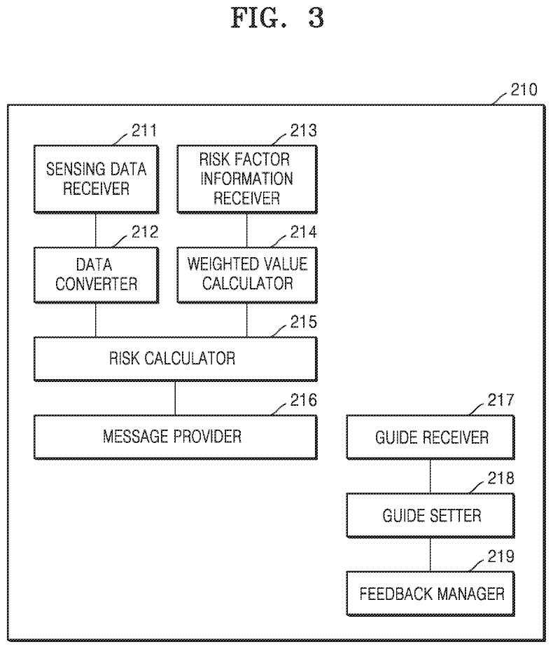

FIG. 3 is a view illustrating a structure of the storage medium 210.

The storage medium 210 may include a sensing data receiver 211, a data converter 212, a risk factor information receiver 213, a weighted value calculator 214, a risk calculator 215, a message provider 216, a guide receiver 217, a guide setter 218, and a feedback manager 219.

The sensing data receiver 211 receives sensing data received through the sensor unit 250. The sensing data receiver 211 retrieves a facial area of the user from a captured image frame and receives first sensing data related to movements of an eyeball (eye) of the user. The sensing data receiver 211 determines a position of the eye existing in the facial area of the user and senses the movements of the eye while monitoring the determined position of the eye. The first sensing data may include the number of blinks for unit time and may selectively include whether the eye is red, an overall shape of the eye, and the like.

The data converter 212 analyzes the received sensing data to convert the sensing data into blink information. The data converter 212 may generate the blink information on the basis of the first sensing data. Here, the blink information may include short-time blink information that is information about the number of blinks within a first reference time on the basis of first sensing data for a preset observation time and long-time blink information that is information about the number of blinks taking longer than or equal to a second reference time for the preset observation time. In addition, the blink information may be determined as a sum of the short-time blink information and the long-time blink information.

The risk factor information receiver 213 receives risk factor information according to a user input that is input through an input/output interface. The risk factor information receiver 213 may receive first risk factor information from the user. The received risk factor information may include age, gender, occupation, sleep time, an electronic device use time for a day, and the like of the user. The risk factor information may further include medical history information of the user and may include biometric information, environmental information, and eye disease information.

The biometric information is about biological information related to the user and includes age, gender, and the like. The environmental information is information about a computer monitor use time, a driving time (daytime, nighttime, or the like), a working environment (dryness, temperature, and the like), and the like for a day. The eye disease information may include a presence or absence of existing diagnosis of dry eye syndrome, use information of eye drops therefor, a history of refractive surgery such as LASIK, LASEK, intraocular lens implantation, and the like, a presence or absence of an eye disease including glaucoma and cataract, and therapy information thereof. The age information may be one selected from 10-20s, 30-40s, 50-60s, and 70s or more, and the gender information may be one selected from male and female. The biometric information may further include medical history information excluding an eye disease. The medical history information may include a history of endocrine therapy such as diabetes, thyroid grand, menopause or the like, therapy information thereof, a presence or absence of anti-cancer therapy and a rheumatic immune metabolic disease, and therapy information thereof.

The weighted value calculator 214 may calculate a weighted value used for calculating a risk of dry eye by using risk factor information. The weighted value calculator 214 calculates one or more first weighted values by using the first risk factor information. The first weighted value is determined by combining one or more pieces of information included in risk factor information about the user. For example, in relation to the risk factor information of the user, a monitor use time longer than or equal to 5 hours, a driving time longer than or equal to 2 hours, night driving longer than or equal to 40 minutes, dryness lower than or equal to reference dryness, a temperature higher than or equal to a reference temperature, a presence or absence of a diagnosis history of dry eye syndrome, a history of LASIK, a high myopia-related history, a glaucoma-related history, 70s, female, diabetes, thyroid grand, hypofunction, menopause, a presence or absence of anti-cancer therapy and an immune disease, and the like may be set to reference risk factor information. An electronic device may classify risk factor information of a user by each factor and compare the classified risk factor information with reference risk factor information. The comparison result may be differently output according to details of the reference risk factor information. For example, the comparison result may include whether risk factor information set to a range is included in the reference risk factor information and whether the set risk factor information is consistent with the reference risk factor information.

The risk calculator 215 may calculate a first risk of dry eye by combining the first weighted value with the blink information. The risk calculator 215 may calculate a risk of dry eye for each period by using a weighted value calculated on the basis of blink information sensed for each period (day, week, month, or the like) and risk factor information for each period. The risk calculator 215 may calculate a risk of dry eye reflecting a current state of the user through blink information and risk factor information acquired in real time.

The message provider 216 may generate a message including the calculated risk of dry eye and provide the message through the input/output device 260. In addition, the message provider 216 may transmit the message including the risk of dry eye to an electronic device of an expert group. Here, the message provider 216 may determine a reference range to which the risk of dry eye belongs. The message provider 216 may transmit the risk of dry eye to an electronic device of an expert group that is randomly selected. The message provider 216 may transmit the message including the risk of dry eye only to an electronic device of an expert group corresponding to the risk of dry eye. In other words, when a risk of dry eye belonging to a first reference range is calculated, the message provider 216 may transmit a message including the risk of dry eye to an electronic device of an expert group corresponding to the first reference range.

The guide receiver 217 may receive a response message including guide information from the electronic device of the expert group that receives the message including the risk of dry eye. The guide information is written in a format and protocol that may be decoded by a device for measuring a risk of dry eye. The guide information may include a longest continuous use time, screen brightness information, rest time information after continuous use, and the like.

The guide setter 218 may set a terminal use restriction according to the guide information. The guide setter 218 may restrict use of a device so that the device is not used in excess of the longest continuous use time according to the guide information. Restricting the use may refer to forcibly stopping an output of the input/output device 260. The guide setter 218 may be embodied so that the screen brightness information is set according to the guide information. The guide setter 218 may be embodied so that the use of the electronic device is restricted after continuous use according to the rest time information after the continuous use of the guide information.

The feedback manager 219 may combine the first risk of dry eye, the first blink information, the first risk factor information, and the guide information into one record. The feedback manager 219 may generate and manage one record by combining the first blink information that is measurement-related information of the risk of dry eye, the first risk factor information, the risk of dry eye that is consequent diagnosis information, and the guide information that is a solution to the diagnosed risk of dry eye. Here, the generated record may be stored without identification information of each user and managed to protect privacy of each user.

When the first risk of dry eye is lower than a second risk of dry eye, the feedback manager 219 may transmit result data including the second risk of dry eye, blink information at a second point in time, and risk factor information at the second point in time to an expert group that generates and transmits guide information.

When the first risk of dry eye is higher than the second risk of dry eye, the feedback manager 219 may generate improvement effect data of the guide information in connection with an improvement effect of the guide information and transmit the improvement effect data to an expert group that is a sender of the guide information or a terminal of the expert group.

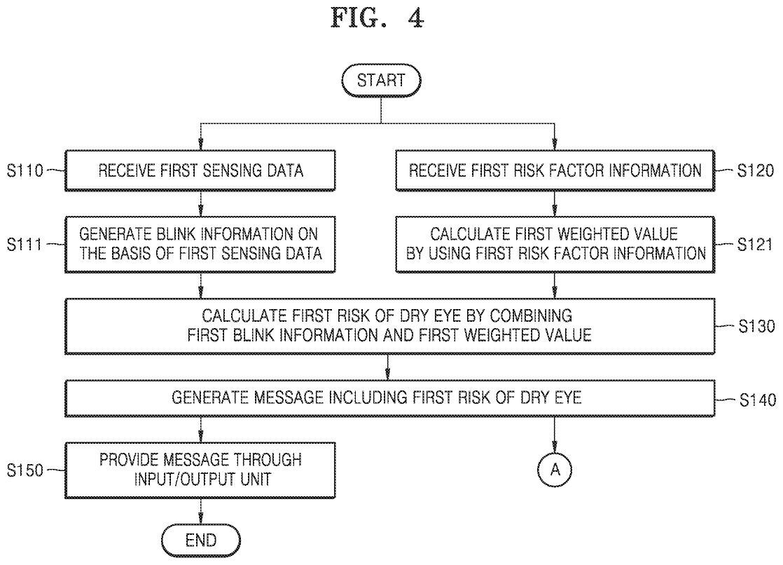

FIGS. 4 through 7 are flowcharts of methods for measuring a risk of dry eye according to aspects of the present disclosure.

As shown in FIG. 4, a method for measuring a risk of dry eye according to an aspect of the present disclosure may include operation S110 receiving first sensing data formed by sensing a set area in a body of a user, operation S120 receiving first risk factor information, operation S111 generating and outputting first blink information related to blinking of the user on the basis of the first sensing data, operation S121 calculating a first weighted value by using the first risk factor information, operation S130 calculating a first risk of dry eye by combining the first blink information and the first weighted value, operation S140 generating a message including the first risk of dry eye, and operation S150 providing the message through an input/output device.

In operation S110, an electronic device searches for a facial area of the user and receives the first sensing data formed by sensing a face of the user for a first time interval. The electronic device determines a position of an eye existing in the facial area of the user and senses movements of the eye while monitoring the determined position of the eye. The first sensing data may include the number of blinks for unit time and may selectively include whether the eye is red, an overall shape of the eye, and the like. When the eye is not detected from the facial area of the user, the electronic device may further perform a process of resetting the facial area. Here, the electronic device may set the facial area to a wider range than a first facial area and detect an eye from the set facial area. Even if an entire area is set to the facial area, when the position of the eye is not detected, the electronic device may provide the user with a notification of resetting the facial area. Selectively, when the position of the eye is not detected, the electronic device stops calculating a risk of dry eye.

In operation S111, the electronic device may generate the blink information on the basis of the first sensing data for the first time interval. Here, the blink information may include short-time blink information that is information about the number of blinks within a first reference time on the basis of first sensing data for a preset observation time and long-time blink information that is information about the number of blinks taking longer than or equal to a second reference time for the preset observation time. In addition, the blink information may be determined as a sum of the short-time blink information and the long-time blink information.

In operation S120, the electronic device may receive, from the user, the first risk factor information related to before the first time interval. Received risk factor information may include age, gender, occupation, a sleep time, an electronic device use time for a day, and the like of the user. The risk factor information may further include medical history information of the user and may include biometric information, environmental information, and eye disease information.

The biometric information is about biological information of the user and includes age, gender, and the like. The environmental information is information about a computer monitor use time for a day, a driving time (daytime, nighttime, or the like), a working environment (dryness, temperature, and the like), and the like. The eye disease information may include a presence or absence of an existing diagnosis of dry eye syndrome, use information of eye drops therefor, a history of refractive surgery such as LASIK, LASEC, intraocular lens implantation, or the like, a presence or absence of an eye disease including glaucoma and cataract, and therapy information thereof. The age information may be one selected from 10-20s, 30-40s, 50-60s, and 70s or more, and the gender information may be one selected from male and female. The biometric information may further include medical history information excluding an eye disease. The medical history information may include a history of endocrine therapy such as diabetes, thyroid gland, menopause, or the like, therapy information thereof, a presence or absence of anti-cancer therapy and a rheumatic immune metabolic disease, and therapy information thereof.

In operation S121, the electronic device calculates one or more first weighted values by using the first risk factor information related to before the first time interval. The first weighted value is determined by combining one or more pieces of information included in risk factor information about the user related to a corresponding time interval. For example, in relation to the risk factor information of the user, a monitor use time longer than or equal to 5 hours, a driving time longer than or equal to 2 hours, night driving longer than or equal to 40 minutes, dryness lower than or equal to reference dryness, a temperate higher than or equal to a reference temperature, a presence or absence of a diagnosis history of dry eye syndrome, a history of LASIK, a history of high myopia, a history of glaucoma, 70s, female, diabetes, thyroid grand, hypofunction, menopause, a presence or absence of anti-cancer therapy and an immune disease, and the like may be set to reference risk factor information. The electronic device may classify the risk factor information of the user by each factor and compare the classified risk factor information with the reference risk factor information. The comparison result may be differently output according to details of the reference risk factor information. For example, the comparison result may include whether reference risk factor information set to a range is included in the reference risk factor information and may include whether the set reference risk factor information is consistent with the reference risk factor information.

In operation S130, the electronic device may calculate the first risk of dry eye by combining the first weighted value with the blink information. The electronic device may calculate a risk of dry eye for each period by using a weighted value calculated on the basis of blink information sensed for each period (day, week, month, or the like) and risk factor information for each period.

In operation S140, the electronic device may generate a message including the first risk of dry eye for the first time interval. Only when the first risk of dry eye exceeds a preset reference risk, the electronic device may generate the message including the first risk of dry eye.

In operation S150, the electronic device may provide the message including the first risk of dry eye.

Therefore, the method for measuring the risk of dry eye according to the aspects of the present disclosure may calculate the risk of dry eye in consideration of information related to blinks of the user and the biometric information, the environmental information, the eye disease information, the medical history information, and the like of the user.

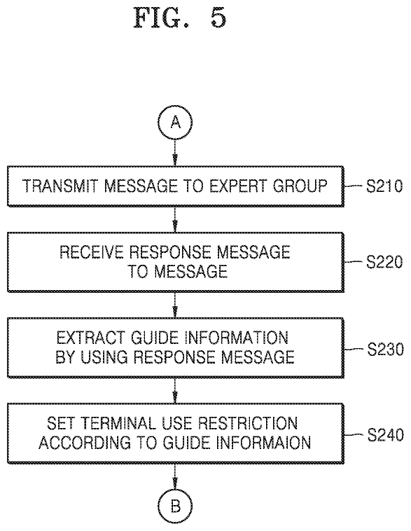

As shown in FIG. 5, the method for measuring the risk of dry eye according to the aspects of the present disclosure may further include operation S210 transmitting the message to the expert group, operation S220 receiving a response message to the message, operation S230 extracting the guide information by using the response message, and operation S240 setting a terminal use restriction according to the guide information.

In operation S210, the electronic device may transmit, to the expert group, the message including a risk of dry eye calculated by comprehensively using blink information including a blink pattern of the user recognized at a current point in time and risk factor information generated on the basis of information about the body, age, and the like of the user that are not directly affected by the eye. Here, the expert group may be determined in consideration of the user and the risk of dry eye of the user and may an ophthalmology-related certificate holder. The transmission to the expert group may be substantially the same as transmission to a user terminal corresponding to the expert group.

In operation S220, the electronic device may receive the response message as a response to the message from the expert group or an electronic device of the expert group.

In operation S230, the electronic device may extract the guide information by using the response message. The guide information is written in a format and protocol that may be decoded by a device for measuring a risk of dry eye. The guide information may include a longest continuous use time, screen brightness information, rest time information after continuous use, and the like.

In operation S240, the electronic device may set the terminal use restriction according to the guide information. The electronic device may restrict use so as not to exceed the longest continuous use time according to the guide information. The electronic device may be embodied so that the screen brightness information is set according to the guide information. The electronic device may be embodied so that use of the electronic device is restricted after continuous use according to the rest time information after the continuous use of the guide information.

Therefore, the method for measuring the risk of dry eye according to the aspects of the present disclosure may receive guide information for lowering a risk of dry eye measured in a current situation of a user from an external expert group and may automatically set the guide information.

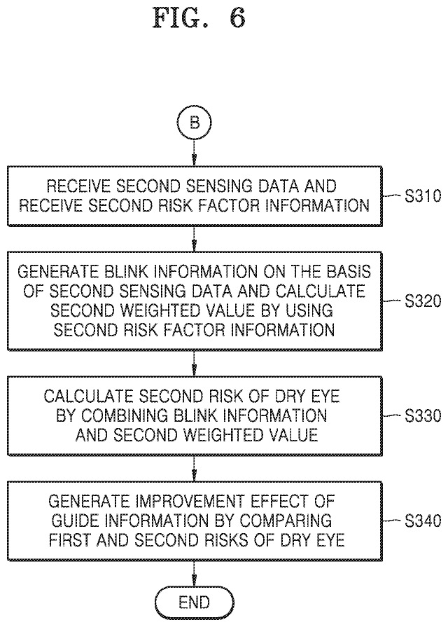

As shown in FIG. 6, according to the aspects of the present disclosure, the method for measuring the risk of dry eye may further include operation S310 receiving second sensing data at a preset point in time after the guide information is set and receiving second risk factor information, operation S320 generating blink information on the basis of the second sensing data and calculating a second weighted value from the second risk factor information including environmental information, habit information, biometric information, and the like after measuring the first risk of dry eye, operation S330 calculating a second risk of dry eye by combining the blink information and the second weighted value, and operation S340 generating an improvement effect of the guide information by comparing the first and second risks of dry eye.

The blink information generated on the basis of the second sensing data may be generated through the process of generating the first blink information, included in the description of FIG. 4. The blink information may be generated by combining short-time blink information and long-time blink information included in the second sensing data.

The improvement effect of the guide information may be calculated on the basis of whether the second risk of dry eye is lower than the first risk of dry eye. For example, when the second risk of dry eye is lower than the first risk of dry eye, the improvement effect of the guide information may be set to a positive value and may be set to a value proportional to a difference value between the first and second risks of dry eye.

In another aspect, the improvement effect of the guide information may be calculated by comparing blink information at a first point in time and blink information at a second point in time. When the blink information at the second point in time is more than the blink information at the first point in time, the improvement effect of the guide information may be set to a negative value and may be set to a value proportional to a difference value between the blink information at the first point in time and the blink information at the second point in time.

The improvement effect of the guide information may be calculated by comparing the first weighted value at the first point in time and the second weighted value at the second point in time. When the second weighted value is smaller than the first weighted value, the improvement effect of the guide information may be set to a positive value and may be set to a value proportional to a difference value between the first and second weighted values.

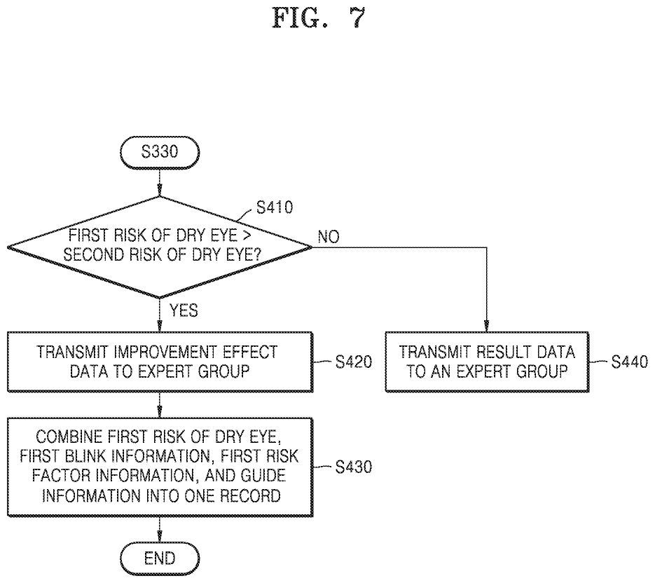

As shown in FIG. 7, according to an aspect of the present disclosure, a process of evaluating the guide information received from the expert group may be performed.

In operation S410, the electronic device may compare the first risk of dry eye and the second risk of dry eye.

When the first risk of dry eye is higher than the second risk of dry eye, the electronic device may generate improvement effect data of the guide information in relation to the improvement effect of the guide information and transmit the improvement effect data to the expert group that is the sender of the guide information or the terminal of the expert group in operation 420.

In operation S430, the electronic device may combine the first risk of dry eye, the first blink information, the first risk factor information, and the guide information into one record. The electronic device may generate and manage one record by combining the first blink information that is measurement-related information of the risk of dry eye, the first blink information, the risk of dry eye that is consequent diagnosis information, and the guide information that is a solution to the diagnosed risk of dry eye. Here, the generated record is stored without identification information of each user and managed to protect privacy of each outer.

When the first risk of dry eye is lower than the second risk of dry eye, the electronic device may transmit, to an expert group that generates and transmits the guide information, result data including the second risk of dry eye, the blink information at the second point in time, and the risk factor information at the second point in time in operation S440. In particular, when the improvement is not made through the guide information of the expert group, for post-management of the risk of dry eye of the user and improvement of the guide information, the device, the method, and the system for measuring the risk of dry eye according to the aspects of the present disclosure generate result data for the guide information received from the expert group and transmits the result data for the guide information to the expert group. The expert group may generate guide information showing an improvement effect by analyzing the guide information and the result data for the guide information.

Therefore, the method for measuring the risk of dry eye according to the aspects of the present disclosure may not only measure the risk of dry eye, but also generate feedback on guide information that is a solution to lower the risk of dry eye. This feedback may affect an evaluation level for an expert group. These measurements, diagnosis, and subsequent therapy effects may also be managed.

The device described above may be embodied as hardware components, software components, and/or a combination of hardware components and software components. For example, the device and elements described in the aspects may be embodied by using one or more general purpose computers or special purpose computers such as a processor, a controller, an arithmetic logic unit (ALU), a digital signal processor, a microcomputer, a field programmable gate array (FPGA), a programmable logic unit (PLU), a microprocessor, or any other devices capable of executing and responding to instructions. A processing unit may execute an operating system (OS) and one or more software applications executed on the OS. In addition, the processing unit may access, store, manipulate, process, and generate data in response to the execution of software. For convenience of description, one processing unit may be described as being used, but one of ordinary skill in the art may understand that the processing unit may include a plurality of processing elements and/or a plurality of types of processing elements. For example, the processing unit may include a plurality of processors or one processor and one controller. Also, the processing unit may include another processing configuration such as a parallel processor.

Software may include a computer program, code, instructions, or a combination of one or more thereof, and configure the processing unit to operate as wanted or independently or collectively instruct the processing unit. Software and/or data may be permanently or temporarily embodied in any type of machine, a component, a physical device, virtual equipment, a computer storage medium or device, or transmitted signal waves to be interpreted by the processing unit or provide the processing unit with instructions or data. The software may be distributed over networked computer systems to be stored or executed in a distributed manner. The software and data may be stored in one or more computer-readable recording media.

The method according to the aspects may be embodied in the form of computer instructions that may be executed through various types of computer means and then recorded in a computer-readable recording medium. The computer-readable recording medium may include program instructions, data files, data structures, and the like alone or in combination. The program instructions recorded on the computer-readable recording medium may be particularly designed and configured for the aspects or may be well known to and used by one of ordinary skill in the computer software art. Examples of the computer-readable recording medium include magnetic media such as a hard disk, a floppy disk, and a magnetic tape, optical media such as CD-ROM and DVD, magneto-optical media such as a floptical disk, and a hardware device particularly configured to store and execute program instructions such as ROM, RAM, flash memory, and the like. Examples of the program instructions include not only machine language codes generated by a compiler, but also high-level language codes that may be executed by a computer by using an interpreter or the like. The hardware device described above may be configured to operate as one or more software modules so as to perform operations of the aspects, and the reverse thereof is the same.

Although the aspects have been described above by limited aspects and drawings, various modifications and changes may be made from the above description by one of ordinary skill in the art. For example, the described techniques may be performed in a different order than the described method, and/or components of the described system, structure, device, circuit, and the like may be combined or joined in a different form than the described method, or even if replaced or substituted by other components or equivalents, an appropriate result may be achieved.

Therefore, other embodiments, other aspects, and equivalents to claims also belong to the scope of claims that will be described below.

What is claimed is:

1. A method of measuring the risk of dry eye, comprising:
receiving, using at least one processor, first sensing data formed by sensing a set area in a body of a user from an electronic device of the user;
receiving, using the at least one processor, first risk factor information related to the user;
outputting, using the at least one processor, first blink information related to blinking of the user, on the basis of the first sensing data;
calculating, using the at least one processor, a first weighted value corresponding to the first risk factor information, on the basis of a relationship between one or more pieces of the received first risk factor information and a risk degree;
calculating, using the at least one processor, a first risk of dry eye of the user by combining the first blink information and the first weighted value, and providing the first risk of dry eye through an input/output device of the electronic device of the user;
determining, using the at least one processor, whether the first risk of dry eye belongs to a first reference range;
on the basis of the determination result, transmitting, using the at least one processor, a request message comprising the first risk of dry eye to an expert group relating to the user or the first reference range;
receiving a response message, which is a response to the request message, comprising guide information configured to reduce the first risk of dry eye from an electronic device of the expert group and providing the response message to the electronic device of the user; and
separating, using the at least one processor, the guide information included in the response message and setting a terminal use restriction according to the guide information.

2. The method of claim 1, wherein the guide information comprises at least one selected from the group consisting of a longest continuous use time, screen brightness information, and rest time information after continuous use.

3. The method of claim 1, further comprising:
after the setting, receiving, using the at least one processor, second sensing data formed by sensing the set area and receiving second risk factor information at a point in time after the terminal use restriction is applied;
calculating, using the at least one processor, a second weighted value corresponding to the second risk factor information;
outputting, using the at least one processor, second blink information related to blinking of the user, on the basis of the second sensing data; and
calculating, using the at least one processor, a second risk of dry eye of the user by combining the second blink information and the second weighted value, generating an improvement effect of the guide information by comparing the first and second risks of dry eye, and providing a message comprising the improvement effect.

4. A device for measuring the risk of dry eye, comprising:
a sensing data receiver configured to receive first sensing data formed by sensing a set area in a body of a user from an electronic device of the user;
a risk factor information receiver configured to receive first risk factor information related to the user;
a data converter configured to output first blink information related to blinking of the user, on the basis of the first sensing data;
a weighted value calculator configured to calculate a first weighted value corresponding to the first risk factor information, on the basis of a relationship between one or more pieces of the received first risk factor information and a risk degree;
a risk calculator configured to calculate a first risk of dry eye of the user by combining the first blink information and the first weighted value; and
a message provider configured to provide a message comprising the first risk of dry eye through an input/output device,
wherein the message provider is configured to:
determine whether the first risk of dry eye belongs to a first reference range, and on the basis of the determination result, transmit a request message comprising the first risk of dry eye to an expert group relating to the user or the first reference range,
receive a response message, which is a response to the request message, comprising guide information configured to reduce the first risk of dry eye from an electronic device of the expert group and provide the response message to the electronic device of the user, and
separate the guide information included in the response message and set a terminal use restriction according to the guide information.

5. A non-transitory computer-readable recording medium storing instructions to execute a method of measuring the risk of dry eye, the method comprising:
receiving, using at least one processor, first sensing data formed by sensing a set area in a body of a user from an electronic device of the user;
receiving, using the at least one processor, first risk factor information related to the user;
outputting, using the at least one processor, first blink information related to blinking of the user, on the basis of the first sensing data;
calculating, using the at least one processor, a first weighted value corresponding to the first risk factor information, on the basis of a relationship between one or more pieces of the received first risk factor information and a risk degree;
calculating, using the at least one processor, a first risk of dry eye of the user by combining the first blink information and the first weighted value, and providing the first risk of dry eye through an input/output device of the electronic device of the user;
determining, using the at least one processor, whether the first risk of dry eye belongs to a first reference range;
on the basis of the determination result, transmitting, using the at least one processor, a request message comprising the first risk of dry eye to an expert group relating to the user or the first reference range;
receiving a response message, which is a response to the request message, comprising guide information configured to reduce the first risk of dry eye from an electronic device of the expert group and providing the response message to the electronic device of the user; and
separating, using the at least one processor, the guide information included in the response message and setting a terminal use restriction according to the guide information.

6. The computer-readable recording medium of claim 5, wherein the guide information comprises at least one selected from the group consisting of a longest continuous use time, screen brightness information, and rest time information after continuous use.

7. The computer-readable recording medium of claim 5, further comprising instructions to execute:
after the setting, receiving, using the at least one processor, second sensing data formed by sensing the set area and receiving second risk factor information at a point in time after the terminal use restriction is applied;
calculating, using the at least one processor, a second weighted value corresponding to the second risk factor information;
outputting, using the at least one processor, second blink information related to blinking of the user, on the basis of the second sensing data; and
calculating, using the at least one processor, a second risk of dry eye of the user by combining the second blink information and the second weighted value, generating an improvement effect of the guide information by comparing the first and second risks of dry eye, and providing a message comprising the improvement effect.
